(19) United States
(12) Patent Application Publication
(10) Pub. No.: US 2007/0178159 A1
(43) Pub. Date: Aug. 2, 2007

(54) In-Situ Forming Porous Scaffold

(75) Inventors: Guohua Chen, Sunnyvale, CA (US); Zhongli Ding, Sunnyvale, CA (US); Johanna (Hanne) Bentz, Newark, CA (US); Paul Houston, Hayward, CA (US); Ling-Ling Kang, Palo Alto, CA (US)

Correspondence Address: DEWIPAT INCORPORATED, P.O. BOX 1017, CYPRESS, TX, 77410-1017, US

(73) Assignee: ALZA CORPORATION, Mountain View, CA (US)

(21) Appl. No.: 11/623,819

(22) Filed: Jan. 17, 2007

Related U.S. Application Data
(60) Provisional application No. 60/763,230, filed on Jan. 30, 2006.

(51) Int. Cl. A61K 38/20; A61K 38/19; A61K 38/18; A61K 9/14

(52) U.S. Cl. 424/484; 424/85.1; 424/85.2; 514/12

(57) ABSTRACT

A composition includes a viscous gel formed from a combination of a biodegradable polymer and a biocompatible solvent. The composition also includes a hydrophilic porogen, which may be incorporated in the viscous gel. The composition may form a porous scaffold in situ.

### CROSS-REFERENCE TO RELATED TO APPLICATIONS

This application claims priority from U.S. provisional application no. 60/763230, filed Jan. 30, 2006, the content of which is incorporated herein by reference.

### BACKGROUND OF THE INVENTION

Porous scaffolds for tissue engineering, such as bone or cartilage regeneration, are usually prefabricated three-dimensional biodegradable polymer structures. Prior art methods for fabricating these fixed porous scaffolds include fiber bonding, solvent casting/particulate leaching, gas foaming, and phase separation/emulsification. (See, for example, Mikos, Antonios G. and Temenoff, Johnna S., “Formation of highly porous biodegradable scaffolds for tissue engineering,” EJB Electronic Journal of Biotechnology, Vol. 3 No. 2, Issue of Aug. 15, 2000.) Prefabricated porous scaffolds require invasive surgery to implant them in anatomical sites. It is also time consuming and inconvenient to reshape prefabricated porous scaffolds to suit a specific patient. Implantation of prefabricated porous scaffolds becomes more difficult if the implant sites have limited access or a complex shape. From the foregoing, a porous scaffold that forms in situ at an anatomical site may offer advantages over a prefabricated porous scaffold.

U.S. Patent Application Publication No. 2002/0193883 describes an injectable implant that includes a bone-like compound, a hydrophobic carrier or degradable component, and optionally an aqueous component. The bone-like compound may include a growth factor, hormone, or protein. The hydrophobic carrier may be selected from polyglycolic acid, copolymer of polycaprolactone and polyglycolic acid, or other polyesters, polyanhydrides, polyamines, nylons, and combinations thereof. The aqueous component may be water, saline, blood, or mixtures thereof. The degradable component may be gelatin, polyglycolic acid and other polyhydroxypolyesters, cross-linked albumin, collagen, proteins, polysaccharides, glycoproteins, or combinations thereof. The mixture of bone-like compound, hydrophobic carrier or degradable component, and aqueous component sets up in situ, leaving a porous implant at the site of need. Subsequently, the hydrophobic carrier or degradable component dissolves or degrades, leaving a bone-like material with interconnected porosity.

### SUMMARY OF THE INVENTION

In one aspect, the invention relates to a composition which comprises a viscous gel formed from a combination of a biodegradable polymer and a biocompatible solvent. The composition further includes a hydrophilic porogen. In one embodiment, the composition forms a porous scaffold in situ.

Other features and advantages of the invention will be apparent from the following description and the appended claims.

### BRIEF DESCRIPTION OF THE DRAWINGS

FIG. 1 is a schematic of an in-situ forming porous scaffold.

FIG. 2 is a cross-section of an in-situ forming porous scaffold after three days in an environment of use.

FIG. 3 illustrates cumulative release of bovine serum albumin (BSA) over time for in-situ forming porous scaffolds.

FIG. 4 is a graph illustrating release rate of BSA over time for in-situ forming porous scaffolds.

FIG. 5 is a graph illustrating co-delivery of multiple proteins from in-situ forming porous scaffolds.

### DETAILED DESCRIPTION OF THE INVENTION

The invention will now be described in detail with reference to a few preferred embodiments, as illustrated in accompanying drawings. In the following description, numerous specific details are set forth in order to provide a thorough understanding of the invention. However, it will be apparent to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow.

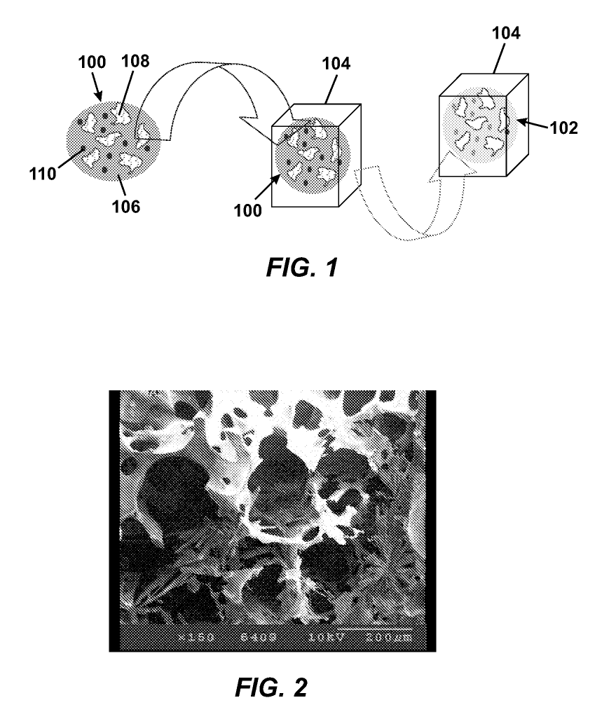

FIG. 1 illustrates an in-situ forming porous scaffold composition 100. The in-situ forming porous scaffold composition 100 forms a porous scaffold 102 at an anatomical site 104. The term “anatomical site” is intended to cover any tissue or organ site where the porous scaffold 102 is desired. The composition 100 includes a viscous gel 106, a porogen 108, and optionally an active agent formulation 110. The composition 100 is preloaded in a reservoir of a delivery device and delivered to the anatomical site 104 using the delivery device. The delivery device may be any suitable device for delivering the composition 100 to the anatomical site 104, such as a cannula, syringe or patch. The porous scaffold 102 is formed in situ at the anatomical site 104. The porous scaffold 102 may be used for tissue engineering, i.e., to aid cell proliferation and adhesion at an anatomical site, or to project injuries, such as bone, burns or scars. The composition 100 is fluidic and can fill any shaped spaces, rendering it suitable for cavities with complex geometry. The composition 100 can provide controlled release of the active agent formulation 110 at the anatomical site 104. In one example, the active agent formulation 110 includes a growth factor or a tissue growth promoting agent, or multiple growth factors to provide synergistic or sequential promotion to tissue growth, and the porous scaffold 102 provides sustained release of the active agent to stimulate tissue regeneration.

The viscous gel 106 includes a biodegradable polymer. The term “biodegradable” means that the polymer gradually decomposes, dissolves, hydrolyzes and/or erodes in situ. Preferably, the biodegradable polymer is also biocompatible. The term “biocompatible” means that the polymer does not cause irritation or necrosis in the environment of use. The viscous gel 106 also includes a biocompatible solvent which combines with the biodegradable polymer to form a viscous gel. Typically, the viscosity of the viscous gel 106 is in a range from 500 poise to 200,000 poise, preferably from about 1,000 poise to about 50,000 poise.

Biodegradable polymers used in the viscous gel 106 typically have molecular weights ranging from about 3,000 to about 250,000. Biodegradable polymer is typically present in the viscous gel 106 in an amount ranging from about 5 to 80% by weight, preferably from about 20 to 70% by weight, more preferably from about 40 to 60% by weight. Examples of biodegradable polymers that are biocompatible include, but are not limited to, polylactides, lactide-based copolymers, polyglycolides, polycaprolactones, polyanhydrides, polyamines, polyesteramides, polyorthoesters, polydioxanones, polyacetals, polyketals, polycarbonates, polyorthocarbonates, polyphosphazenes, succinates, poly(malic acid), poly(amino acids), polyphosphoesters, polyesters, polybutylene terephthalate, and copolymers, terpolymers and mixtures thereof.

In one example, the biodegradable polymer used in the viscous gel 106 is a lactide-based polymer. A lactide-based polymer is a copolymer of lactic acid and glycolic acid. The lactide-based polymer can include small amounts of other comonomers that do not substantially affect the advantageous results that can be achieved in accordance with the invention. The term “lactic acid” includes the isomers L-lactic acid, D-lactic acid, DL-lactic acid, and lactide. The term “glycolic acid” includes glycolide. The polymer may have a lactic-acid to glycolic-acid monomer ratio of from about 100:0 to 15:85, preferably from about 60:40 to 75:25, often about 50:50. The polylactide polymer may have a number average molecular weight ranging from about 1,000 to about 120,000, preferably from about 5,000 to about 30,000, as determined by gel permeation chromatography.

Examples of commercially-available biodegradable polymers include, but are not limited to, Poly D,L-lactide, available as RESOMER® L 104, RESOMER® R 104, RESOMER® 202, RESOMER® 203, RESOMER® 206, RESOMER® 207, RESOMER® 208; Poly D,L-lactide-co-glycolide (PLGA), L/G ratio of 50/50, available as RESOMER® RG 502H; PLGA, L/G ratio of 50/50, available as RESOMER® RG 503; PLGA, L/G ratio of 50/50, available as RESOMER® RG 755; Poly L-lactide, molecular weight of 2000, available as RESOMER® L 206, RESOMER® L 207, RESOMER® L 209, RESOMER® L 214; Poly L-lactide-co-D,L-lactide, L/G ratio of 90/10, available as RESOMER® LR 209; PLGA, L/G ratio of 75/25, available as RESOMER® RG 752, RESOMER® RG 756, PLGA, L/G ratio of 85/15, available as RESOMER® RG 858; Poly L-lactide-co-trimethylene carbonate, L/G ratio of 70/30, available as RESOMER® LT 706, and Poly dioxanone, available as RESOMER® X210 (Boehringer Ingelheim Chemicals, Inc. Petersburg, Va.).

Additional examples of commercially-available biodegradable polymers include, but are not limited to, DL-lactide/glycolide (DL), L/G ratio of 100/0, available as MEDISORB® Polymer 100 DL High, MEDISORB® Polymer 100 DL Low; DL-lactide/glycolide (DL), L/G ratio of 85/15, available as MEDISORB® Polymer 8515 DL High, MEDISORB® Polymer 8515 DL Low; DL-lactide/glycolide (DL), L/G ratio of 75/25, available as MEDISORB® Polymer 7525 DL High, MEDISORB® Polymer 7525 DL Low; DL-lactide/glycolide (DL), L/G ratio of 65/35, available as MEDISORB® Polymer 6535 DL High, MEDISORB® Polymer 6535 DL Low; DL-lactide/glycolide (DL), L/G ratio of 54/46, available as MEDISORB® Polymer 5050 DL High, MEDISORB® Polymer 5050 DL Low, MEDISORB® 5050 Polymer DL 2A(3), MEDISORB® 5050 Polymer DL 3A(3), MEDISORB® 5050 Polymer DL 4A(3) (Medisorb Technologies International L.P., Cincinnati, Ohio).

Additional examples of commercially-available biodegradable polymers include, but are not limited to, PLGA (L/G ratio of 50/50), PLGA (L/G ratio of 65/35), PLGA (L/G ratio of 75/25), PLGA (L/G ratio of 85/15), Poly D,L-lactide, Poly L-lactide, Poly glycolide, Poly ε-caprolactone, Poly D,L-lactide-co-caprolactone (L/C ratio of 25/75), and Poly D,L-lactide-co-caprolactone (L/C ratio of 75/25), available from Birmingham Polymers, Inc., Birmingham, Ala.

The solvent used in the viscous gel 106 is typically an organic solvent and may be a single solvent or a mixture of solvents. To limit water uptake by the viscous gel 106 in the environment of use, the solvent, or at least one of the components of the solvent in the case of a multi-component solvent, should have limited miscibility with water, e.g., less than 7% by weight, preferably less than 5% by weight, more preferably less than 3% by weight miscibility with water. In one example, the viscous gel 106 includes one or more hydrophobic solvents selected from aromatic alcohols, the lower alkyl and aralkyl esters of aryl acids such as benzoic acid, the phthalic acids, salicylic acid, lower alkyl esters of citric acid, such as triethyl citrate and tributyl citrate and the like, and aryl, aralkyl and lower alkyl ketones.

In one example, the solvent used in the viscous gel 106 is selected from aromatic alcohols having the following structural formula:
Ar-(L)ₙ-OH (1)
In the formula above, Ar is a substituted or unsubstituted aryl or heteroaryl group, n is zero or 1, and L is a linking moiety. Preferably, Ar is a monocyclic aryl or heteroaryl group, optionally substituted with one or more non-interfering substituents such as hydroxyl, alkoxy, thio, amino, halo, and the like. More preferably, Ar is an unsubstituted 5- or 6-membered aryl or heteroaryl group such as phenyl, cyclopentadienyl, pyridinyl, pyrimadinyl, pyrazinyl, pyrrolyl, pyrazolyl, imidazolyl, furanyl, thiophenyl, thiazolyl, isothiazolyl, or the like. The subscript “n” is zero or 1, meaning that the linking moiety L may or may not be present. Preferably, n is 1 and L is generally a lower alkylene linkage such as methylene or ethylene, wherein the linkage may include hetero-atoms such as O, N or S. Most preferably, Ar is phenyl, n is 1, and L is methylene, such that the aromatic alcohol is benzyl alcohol.

In another example, the solvent used in the viscous gel is selected from lower alkyl and aralkyl esters of aromatic acids, generally, but not necessarily, having the structural formula: 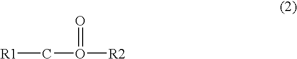
In the formula above, R1 is substituted or unsubstituted aryl, aralkyl, heteroaryl or heteroaralkyl, preferably substituted or unsubstituted aryl or heteroaryl, more preferably monocyclic or bicyclic aryl or heteroaryl optionally substituted with one or more non-interfering substituents such as hydroxyl, carboxyl, alkoxy, thio, amino, halo, and the like, still more preferably 5- or 6-membered aryl or heteroaryl such as phenyl, cyclopentadienyl, pyridinyl, pyrimadinyl, pyrazinyl, pyrrolyl, pyrazolyl, imidazolyl, furanyl, thiophenyl, thiazolyl, or isothiazolyl, and most preferably 5- or 6-membered aryl. R2 is hydrocarbyl or heteroatom-substituted hydrocarbyl, typically lower alkyl or substituted or unsubstituted aryl, aralkyl, heteroaryl or heteroaralkyl, preferably lower alkyl or substituted or unsubstituted aralkyl or heteroaralkyl, more preferably lower alkyl or monocyclic or bicyclic aralkyl or heteroaralkyl optionally substituted with one or more non-interfering substituents such as hydroxyl, carboxyl, alkoxy, thio, amino, halo, and the like, still more preferably lower alkyl or 5- or 6-membered aralkyl or heteroaralkyl, and most preferably lower alkyl or 5- or 6-membered aryl optionally substituted with one or more additional ester groups having the structure —O—(CO)—R1. Most preferred esters are benzoic acid and phthalic acid derivatives.

In yet another example, the solvent used in the viscous gel 106 is selected from aryl and aralkyl ketones generally, but not necessarily, having the structural formula: 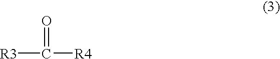
In the formula above, R3 and R4 may be selected from any of the R1 and R2 groups previously described.

Preferred solvents for use in the viscous gel 106 include aromatic alcohols and the lower alkyl and aralkyl esters of aryl acids described above. Representative acids are benzoic acid and the phthalic acids, such as phthalic acid, isophthalic acid, and terephathalic acid. More preferred solvents are benzyl alcohol and derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with benzyl benzoate being most preferred.

Benzoic acid derivatives that may be used in the viscous gel 106 include, but are not limited to, 1,4-cyclohexane dimethanol dibenzoate, diethylene glycol dibenzoate, dipropylene glycol dibenzoate, polypropylene glycol dibenzoate, propylene glycol dibenzoate, diethylene glycol benzoate and dipropylene glycol benzoate blend, polyethylene glycol (200) dibenzoate, isodecyl benzoate, neopentyl glycol dibenzoate, glyceryl tribenzoate, pentaerylthritol tetrabenzoate, cumylphenyl benzoate, trimethyl pentanediol dibenzoate.

Phthalic acid derivatives that may be used in the viscous gel 106 include, but are not limited to, alkyl benzyl phthalate, bis-cumyl-phenyl isophthalate, dibutoxyethyl phthalate, dimethyl phthalate, dimethyl phthalate, diethyl phthalate, dibutyl phthalate, diisobutyl phthalate, butyl octyl phthalate, diisoheptyl phthalate, butyl octyl phthalate, diisononyl phthalate, nonyl undecyl phthalate, dioctyl phthalate, di-isooctyl phthalate, dicapryl phthalate, mixed alcohol phthalate, di-(2-ethylhexyl) phthalate, linear heptyl, nonyl, phthalate, linear heptyl, nonyl, undecyl phthalate, linear nonyl phthalate, linear nonyl undecyl phthalate, linear dinonyl, didecyl phthalate (diisodecyl phthalate), diundecyl phthalate, ditridecyl phthalate, undecyldodecyl phthalate, decyltridecyl phthalate, blend (50/50) of dioctyl and didecyl phthalates, butyl benzyl phthalate, and dicyclohexyl phthalate.

Many of the solvents useful in the invention are available commercially (e.g., from Aldrich Chemicals and Sigma Chemicals) or may be prepared by conventional esterification of the respective arylalkanoic acids using acid halides, and optionally esterification catalysts, such as described in U.S. Pat. No. 5,556,905, which is incorporated herein by reference, and in the case of ketones, oxidation of their respective secondary alcohol precursors.

The viscous gel 106 may include, in addition to the hydrophobic solvent(s) described above, one or more hydrophilic solvents (“component solvents”), provided that any such hydrophilic solvent is other than a lower alkanol. Component solvents compatible and miscible with the primary hydrophobic solvent(s) may have a higher miscibility with water without significantly increasing water uptake by the viscous gel. Such mixtures will be referred to as “component solvent mixtures.” Useful component solvent mixtures may exhibit solubilities in water greater than the primary solvents themselves, typically between 0.1% by weight and up to and including 50% by weight, preferably up to and including 30% by weight, and most preferably up to and including 10% by weight, without significantly increasing water uptake by the viscous gel.

Component solvents useful in component solvent mixtures are those solvents that are miscible with the primary solvent or solvent mixture and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and mixtures thereof.

Preferred solvent mixtures are those in which benzyl benzoate is a primary solvent, and those formed of benzyl benzoate and a component solvent selected from triacetin, tributyl citrate, triethyl citrate or N-methyl-2-pyrrolidone, or glycofurol. Preferred solvent mixtures are those in which benzyl benzoate is present by weight in an amount of 50% or more, more preferably 60% or more, and most preferably 80% or more of the total amount of solvent present. Especially preferred mixtures are those of 80:20 mixtures by weight of benzyl benzoate/triacetin and benzyl benzoate/N-methyl-2-pyrrolidone. In additional examples, the primary solvent is benzyl alcohol, and mixtures formed of benzyl alcohol and either benzyl benzoate or ethyl benzoate. Preferred mixtures of benzyl alcohol/benzyl benzoate and benzyl alcohol/ethyl benzoate are 1/99 mixtures by weight; 20/80 mixtures by weight; 30/70 mixtures by weight; 50/50 mixtures by weight; 70/30 mixtures by weight; 80/20 mixtures by weight; 99/1 mixtures by weight. Especially preferred mixtures of benzyl alcohol/benzyl benzoate and benzyl alcohol/ethyl benzoate are 25/75 mixtures by weight and 75/25 mixtures by weight.

The porogen 108 is selected such that it imparts porosity to the porous scaffold 102 in situ by leaching. The size of the porogen 108 particles typically controls the size of the pores formed in the porous scaffold 102. The pore size may be between 1 μm to about 1000 μm, preferably between 5 μm and 500 μm, most preferably between 30 μm and 300 μm. The pore density may be in a range from 1% to 70% of the total mass of the composition 100, preferably in a range from 5% to 50% of the total mass of the composition 100, more preferably in a range from 10% to 40% of the total mass of the composition 100.

The porogen 108 included in the composition 100 may be selected from the group consisting of sugars, hydrophilic solid polymers, inorganic salts, and hydrogels. The porogen 108 may optionally include a mineral, such as tricalcium phosphate (TCP) to better mimic a bone-like material when applied for bone growth.

Examples of sugars suitable for use as the porogen 108 include, but are not limited to, mannitol, sucrose, trehalose, and sorbitol.

Examples of inorganic salts suitable for use as the porogen 108 include, but are not limited to, sodium chloride, calcium chloride, sodium carbonate, zinc carbonate, magnesium carbonate, calcium carbonate, magnesium hydroxide, calcium hydrogen phosphate, calcium acetate, calcium hydroxide, calcium lactate, calcium maleate, calcium oleate, calcium oxalate, calcium phosphate, magnesium acetate, magnesium hydrogen phosphate, magnesium phosphate, magnesium lactate, magnesium maleate, magnesium oleate, magnesium oxalate, zinc acetate, zinc hydrogen phosphate, zinc phosphate, zinc lactate, zinc maleate, zinc oleate, and zinc oxalate.

Examples of hydrophilic solid polymers for use as the porogen 108 include, but are not limited to, polyethylene glycol, typically with molecular weight between 1,000 and 50,000, block copolymers of ethylene glycol-co-propylene glycol-co-ethylene glycol such as PLURONIC® F68 and F127, polyvinyl pyrrolidone, typically having molecular weight of 1,000 to 50,000, polyvinyl alcohol, polyacrylate, polyethyleneimine, cellulose and its derivatives, fibrin glue, collagen, gelatin, hyaluronic acid, alginate, chitosan derivatives, and other biopolymers.

Hydrogels are water-swollen networks of hydrophilic homopolymers and copolymers. These networks may be formed by various techniques. One common synthetic route is the free radical polymerization of vinyl monomers in the presence of a difunctional crosslinking agent and a swelling agent. Examples of such hydrogels can be polyacrylamide, polyacrylic acid, polyhydroxyethyl mathacrylate (polyHEMA), and polyvinylpyrrolidone. Another way to make cross-linked hydrogel is to react the functional groups in the polymer with a difunctional cross-linking agent in water. One such example is collagen cross-linked with glutaric dialdehyde or multi-functional PEG. Similar cross-linked hydrogels can be made with other proteins and natural polymers such as hyaluronic acid and chitoson. For use as the porogen 108, the hydrogel would be made and dried prior to loading into the viscous gel 106. The particle size and porosity of the hydrogel can be made during the cross-linking reactions.

The active agent formulation 110 included in the composition includes an active agent and may further include excipients to make a stable active agent formulation. For example, the excipients may be selected from the group consisting of sugars, buffers, surfactants, permeation enhancers, and combinations thereof. The invention is not limited by the type of active agent or combination of active agents included in the active agent formulation 110. In one example, the active agent is a growth factor or tissue growth promoting agent. The active agent may be selected from follicle-stimulating hormone, atrial natriuretic factor, filgrastim, epidermal growth factors, platelet-derived growth factor, insulin-like growth factors, fibroblast-growth factors, transforming-growth factors including bone morphogenetic proteins and growth differentiating factors, interleukins, colony-stimulating factors, interferons, endothelial growth factors, erythropoietins, angiopoietins, placenta-derived growth factors, hypoxia induced transcriptional regulators, and human growth hormone.

Release of the active agent may be controlled, for example, by chelating the agent to a metal. The preferred molar ratio for the protein/active agent-metal complex is about 1 to about 0.5 Molar, and/or 1 to about 100 Molar. In one aspect, control of the active agent may be accomplished by placing the active agent in hydrophobic microspheres.

### EXAMPLE 1

Viscous gels having the compositions shown in Table 1 were prepared. The preparation involved taring a glass vessel on a Mettler PJ3000 top loader balance. A biodegradable polymer was added to the glass vessel, followed by a corresponding biocompatible solvent. In this example, the biodegradable polymer was poly D,L-lactide-co-glycolide (PLGA), (L/G ratio of 75/25), available as RESOMER® RG 752 (PLGA-752), and the biocompatible solvent was selected from benzyl benzoate, benzyl alcohol, and mixtures thereof. The polymer/solvent mixture was manually stirred in the glass vessel with a stainless steel square-tip spatula, resulting in a sticky amber paste-like substance containing white polymer particles. The glass vessel with the polymer/solvent mixture was sealed and placed in a temperature controlled incubator equilibrated to 39° C. The polymer/solvent mixture was removed from the incubator when it appeared to be a clear amber homogeneous gel. Incubation time intervals ranged from 1 to 4 days, depending on solvent and polymer type and solvent and polymer ratios.

TABLE 1

| | | BENZYL | BENZYL |
|---|---|---|---|
| FORMULATION | PLGA | BENZOATE | ALCOHOL |
| 1 | 50.0% | 44.8% | 5.1% |
| 2 | 55.0% | 45.0% | |
| 3 | 50.0% | 50.0% | |
| 4 | 45.0% | 55.0% | |

### EXAMPLE 2

Lyophilized bovine serum albumin (BSA), available from Sigma, was grinded. The ground lyophilized BSA was sieved through a 120 mesh screen, followed by a 400 mesh screen, to obtain particles having a size range between 38-125 μm.

### EXAMPLE 3

Porogen particles having the compositions shown in Table 2 were prepared. Porogens were selected from mannitol, sucrose, tricalcium powder, available from Berkeley Advanced Biomaterials Inc., Berkeley, Calif., and mixtures thereof, and blended in a Waring blender. The mixture was then transferred to a 13-mm round compression die and compressed at 5 toms for 5 minutes to form a pellet. The pellet was ground using a Waring blender. Particles were collected between 120-mesh (125 μm) and 400-mesh (300 μm) sieves.

TABLE 2

| FORMULATION | MANNITOL | SUCROSE | TCP |
|---|---|---|---|
| 5 | 100 | 0 | 0 |
| 6 | 75 | 0 | 25 |
| 7 | 25 | 0 | 75 |
| 8 | 0 | 100 | 0 |
| 9 | 0 | 75 | 25 |
| 10 | 0 | 25 | 75 |

### EXAMPLE 4

In situ forming porous scaffold formulations having the compositions shown in Table 3 were prepared. The preparation involved loading BSA particles as prepared in EXAMPLE 2 and porogen particles as prepared in EXAMPLE 3 into viscous gels as prepared in EXAMPLE 1. The BSA particles and viscous gel were initially blended manually until the BSA particles were wetted completely. The resulting mixture was then thoroughly blended by conventional mixing using a Caframo mechanical stirrer with an attached square-tip metal spatula. After a homogeneous mixture was obtained, the porogen particles as prepared in EXAMPLE 3 were added to the mixture. Then, the mixture was again thoroughly blended by conventional mixing using the Caframo mechanical stirrer. Final homogeneous formulations were transferred to 10 cc disposable syringes for storage or dispensing.

TABLE 3

| | | | | BSA |
|---|---|---|---|---|
| | | POROGEN | VISCOUS GEL | PARTICLE |
| | | PARTICLE | (Formulation 4 | LOADING |
| | POROGEN TYPE | LOADING | in Table 1) (vol | (mg/ml |
| FORMULATION | (See Table 2) | (vol %) | %) | scaffold) |
| 11 | N/A | 0 | 100 | 0 |
| 12 | N/A | 0 | 100 | 1.25 |
| 13 | Formulation 5 | 20 | 80 | 0 |
| 14 | Formulation 5 | 30 | 70 | 0 |
| 15 | Formulation 5 | 35 | 65 | 0 |
| 16 | Formulation 5 | 20 | 80 | 1.25 |
| 17 | Formulation 5 | 30 | 70 | 1.25 |
| 18 | Formulation 5 | 35 | 65 | 1.25 |
| 19 | Formulation 5 | 20 | 80 | 1.25 |
| 20 | Formulation 6 | 30 | 70 | 1.25 |
| 21 | Formulation 6 | 35 | 65 | 1.25 |

### EXAMPLE 5

The in-situ forming porous scaffold formulations prepared in EXAMPLE 4 were immersed in sodium phosphate buffer solution (PBS) containing 20% bovine serum for three days or longer and frozen immediately after removing the solution. Cross-sections of the scaffolds were observed on a cold stage with Scanning Electron Microscopy (SEM). The scaffolds were also examined with a light microscope after brief exposure to blue dye. FIG. 2 shows that pores formed in Formulation 13 (see Table 3) within three days of injection into PBS/20% serum solution. The SEM image also shows that the pore size can be as large as ca 300 μm.

### EXAMPLE 6

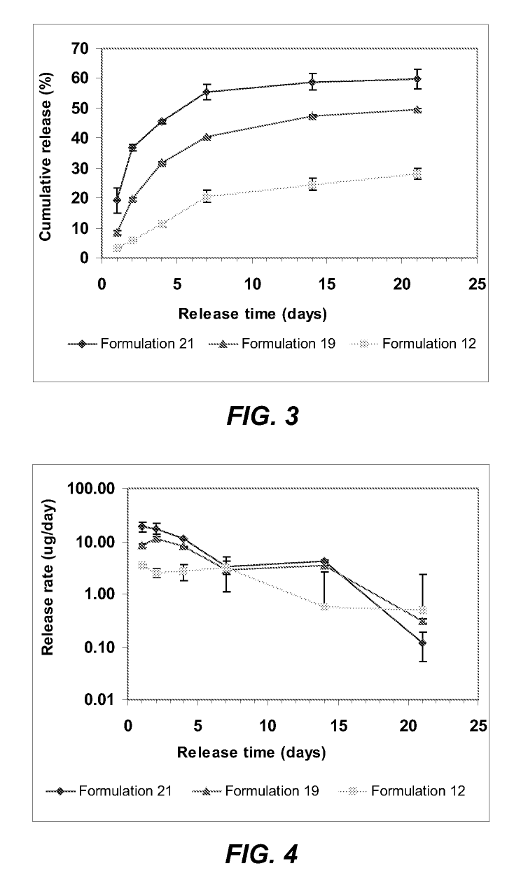

The prepared formulations, as shown in EXAMPLE 4, were injected into pouches made of Millipore membranes. The pouches were then heat sealed and placed in an in-vitro release medium, which is sodium phosphate buffer containing 0.1% TWEEN® 20, at 37° C. The release rates of BSA from the scaffolds were determined by analyzing BSA concentration within the release medium periodically using High Performance Liquid Chromatography (HPLC). FIG. 3 shows percent cumulative release of BSA from Formulations 12, 19, and 21 (see Table 3) over 21 days. FIG. 4 shows release rate (μg/day) of BSA from Formulations 12, 19, and 21 over 21 days. The results show that porogen content affects the release profiles of BSA. In general, the higher the porogen content, the faster BSA was released, but still in a sustained manner. For example, sustained release of BSA from Formulation 18 (see Table 3) was observed for over three weeks even through this formulation contained 35% by volume porogen.

### EXAMPLE 7

A stable solution of rhGDF-5 protein was prepared. RhGDF-5 protein was initially dissolved in 0.01 M HCl. Buffer exchange procedure was performed so that the final solution contained 9 mg/mL rhGDF-5, 36 mg/ml trehalose, 10 mM tris buffer, and 5 mM SDS, 0.02% TWEEN® 80 and 5 mM ethylenediaminetetraacetate (EDTA).

### EXAMPLE 8

RhGDF-5 solution as prepared in EXAMPLE 7 was lyophilized using the dry cycles shown in Table 4. The lyophilized rhGDF-5 was ground and sieved through a 120 mesh screen followed by a 400 mesh screen to obtain stable rhGDF-5 particles having a size range between 38-125 μm.

TABLE 4

| | | | HOLDING |
|---|---|---|---|
| RAMP RATE | TEMPERATURE | VACUUM | TIME |
| (° C./min) | (° C.) | (mτ) | (min) |
| 2.5 | 4 | | 60 |
| 2.5 | −50 | | 180 |
| 0.5 | −15 | 50 | 1440 |
| 0.5 | −5 | 50 | 720 |
| 0.5 | 0 | 200 | 720 |
| 0.2 | 4 | 200 | 5000 |

### EXAMPLE 9

In-situ forming porous scaffold formulations having the compositions shown in Table 5 were prepared using the rhGDF-5 particles prepared as described in EXAMPLE 8, the porogen particles prepared as described in EXAMPLE 3, and the viscous gels prepared as described in EXAMPLE 1. The formulations were prepared as follows: the rhGDF-5 particles and the viscous gel were blended manually until the dry particles were wetted completely. Then, the milky light yellow particle/gel mixture was thoroughly blended by conventional mixing using a Caframo mechanical stirrer with an attached square-tip metal spatula. After a homogenous depot formulation was obtained, porogen particles were added. The mixture was blended manually until the porogen particles were wetted completely. Then, the particle/gel mixture was thoroughly blended by conventional mixing using a Caframo mechanical stirrer with an attached square-tip metal spatula. Final homogenous depot formulations were transferred to 10 cc disposable syringes for storage or dispensing.

TABLE 5

| | TYPE OF | | | VISCOUS GEL |
|---|---|---|---|---|
| | POROGEN | | | (Formulation 2 in |
| FORMU- | (See | rhGDF-5 | POROGEN | Table 1) |
| LATION | Table 2) | (mg) | (g) | (g) |
| 22 | Formulation 5 | 28.3 | 4.59 | 8.57 |
| 23 | Formulation 6 | 28.4 | 4.60 | 8.59 |

### EXAMPLE 10

The in-situ forming porous scaffold formulations prepared as described in EXAMPLE 9 were implanted and evaluated using a cranial defect rat model. The cranial defect was created in the skulls of male Sprague Dawley rats, weighing 180-200 g at the time of surgery. The created defect was 3×5 mm in size. Each defect was filled with one test formulation. Calvariae was retrieved 28 days post surgery from all animals. The calvariae defects were collected for histological evaluation. From the evaluation, porogen with a bone-like mineral TCP appeared to have better bone growth than one without TCP.

### EXAMPLE 11

HGH-Zn particles were prepared. The preparation was as follows: hGH solutions of 40 mg/mL and zinc acetate of 27.2 mM were prepared in 5 mM TRIS buffer, pH 7.0, respectively. A 15:1 final Zn:hGH mole ratio was obtain by mixing equal parts of hGh and zinc acetate solutions together. This solution was allowed to complex for approximately one hour at 4° C. This complex was pre-cooled to −70° C.

### EXAMPLE 12

Lyophilized particles were prepared from hGH formulation solutions as prepared in EXAMPLE 11 using a Durastop μP Lyophilizer in accordance with the freezing and drying cycles shown in Table 6 below. The lyophilized hGH/Zn complex was ground using a Waring blender. Particles were collected between a 120-mesh (125 μm) and 400-mesh (38 μm) sieve (Formulation 24).

TABLE 6

| | |
|---|---|
| Freezing | Ramp down at 2.5 C/min to −1° C. and hold for 30 min |
| cycle | Ramp down at 2.5 C/min to −50° C. and hold for 120 min |
| Drying cycle | Ramp up at 0.16 C/min to −10° C. and hold for 240 min |
| | Ramp up at 0.16 C/min to 0° C. and hold for 720 min |
| | Ramp up at 0.16 C/min to 10° C. and hold for 120 min |
| | Ramp up at 0.16 C/min to 20° C. and hold for 300 min |
| | Ramp up at 0.16 C/min to 30° C. and hold for 300 min |
| | Ramp up at 0.16 C/min to 4° C. and hold for 9000 min |

### EXAMPLE 13

Preparation of in-situ forming scaffold containing multiple proteins: Porogen particles, BSA particles as prepared in EXAMPLE 2, and hGH/Zn particles as prepared in EXAMPLE 12, were loaded into viscous gels as prepared in EXAMPLE 1. The composition of the in-situ forming porous scaffold (Formulation 25) is shown in Table 7 below. The active agent particles (BSA, hGH/Zn) and the viscous gel were blended manually until the dry particles were wetted completely. Then, the milky light yellow particle/gel mixture was thoroughly blended by conventional mixing using a Caframo mechanical stirrer with an attached square-tip metal spatula. After a homogenous depot formulation was obtained, porogen particles were added. The mixture was blended manually until the porogen particles were wetted completely. Then, the particle/gel mixture was thoroughly blended by conventional mixing using a Caframo mechanical stirrer with an attached square-tip metal spatula. Final homogenous depot formulations were transferred to 10 cc disposable syringes for storage or dispensing.

TABLE 7

| | |
|---|---|
| BSA (mg/ml scaffold) | 1.5 |
| HGH/Zn (Formulation 24) (mg/ml scaffold) | 1.5 |
| Porogen (Formulation 5 in Table 2) (vol %) | 30 |
| Viscous gel (Formulation 4 in Table 1) (vol %) | 70 |

### EXAMPLE 14

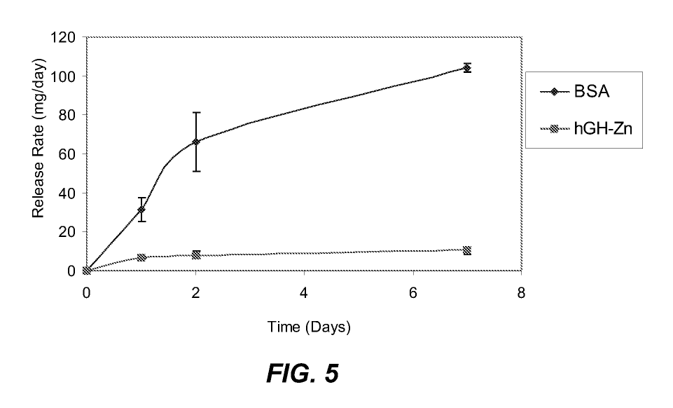

Formulation 25 as described in EXAMPLE 13 was injected into a pouch made of Millipore membranes. The pouch was then heat sealed and placed in an in vitro release medium, which is sodium phosphate buffer containing 0.1% TWEEN® 20, at 37° C. The release rates of BSA and hGH from the scaffold was determined by analyzing BSA and hGH concentrations within the release medium periodically using HPLC. FIG. 5 shows the release profiles of BSA and hGH from the scaffold. The release rate of hGH is significantly slower than that of BSA. This demonstrates that the in-situ forming porous scaffold is able to deliver multiple proteins (growth factors) simultaneously with different release rates. The release rate of individual active agent can be tailored by controlling the active agent particle properties, such as solubility, to deliver the desired amount of each growth factor to provide sufficient stimulation at the stage of tissue growth.

While the invention has been described with respect to a limited number of embodiments, those skilled in the art, having benefit of this disclosure, will appreciate that other embodiments can be devised which do not depart from the scope of the invention as disclosed herein.

What is claimed is:

1. A composition, comprising:
a viscous gel formed from a combination of a biodegradable polymer and a biocompatible solvent; and
a hydrophilic porogen.

2. The composition of claim 1, wherein the hydrophilic porogen is incorporated in the viscous gel.

3. The composition of claim 1, further comprising at least one active agent incorporated in the viscous gel.

4. The composition of claim 3, wherein the active agent comprises a protein.

5. The composition of claim 3, wherein the active agent comprises a growth factor.

6. The composition of claim 3, wherein the active agent comprises a tissue growth promoting agent.

7. The composition of claim 3, wherein the active agent is in a formulation comprising one or more excipients.

8. The composition of claim 3, wherein the active agent is selected from the group consisting of follicle-stimulating hormone, atrial natriuretic factor, filgrastim, epidermal growth factors, platelet-derived growth factor, insulin-like growth factors, fibroblast-growth factors, transforming-growth factors including bone morphogenetic proteins and growth differentiating factors, interleukins, colony-stimulating factors, interferons, endothelial growth factors, erythropoietins, angiopoietins, placenta-derived growth factors, hypoxia induced transcriptional regulators, hypoxia induced transcriptional regulators, or cell adhesion factors, atrial natriuretic factors and human growth hormone, and combinations thereof.

9. A composition according to any of the preceding claims, which is suitable for controlled release of the active agent.

10. The composition of claim 9, which is injectable into an anatomical site.

11. The composition of claim 1, wherein the active agent formulation comprises a plurality of active agents and the composition provides controlled release of each of the active agents at a predetermined rate.

12. The composition of claim 1, wherein the biodegradable polymer is a lactide-based polymer.

13. The composition of claim 1, wherein the biodegradable polymer is selected from the group consisting of polylactides, polyglycolides, polycaprolactones, polyanhydrides, polyamines, polyesteramides, polyothoesters, polydioxanones, polyacetals, polyketals, polycarbonates, polyorthocarbonates, polyphosphazenes, succinates, poly(malic acid), poly(amino acids), polyphosphoesters, polyesters, polybutylene terephthalate, and copolymers, terpolymers and mixtures thereof.

14. The composition of claim 1, wherein the biocompatible solvent comprises one or more hydrophobic solvents.

15. The composition of claim 14, wherein the biocompatible solvent optionally comprises one or more hydrophilic solvents compatible and miscible with the one or more hydrophobic solvents.

16. The composition of claim 15, wherein the hydrophobic component is selected from the group consisting of aromatic alcohols, lower alkyl and aralkyl esters of aryl acids, lower alkyl esters of citric acid and aryl, aralkyl and lower alkyl ketones, and combinations thereof.

17. The composition of claim 16, wherein the hydrophilic component is selected from the group consisting of triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and combinations thereof.

18. The composition of claim 15, wherein the hydrophobic component is selected from the group consisting of aromatic alcohols.

19. The composition of claim 15, wherein the hydrophobic component is selected from the group consisting of phthalic acid, benzoic acid, and salicylic acid.

20. The composition of claim 1, wherein the biocompatible solvent comprises a primary solvent selected from the group consisting of benzyl benzoate, benzyl alcohol, and combinations thereof.

21. The composition of claim 20, wherein the biocompatible solvent further comprises a secondary solvent selected from the group consisting of triacetin, tributyl citrate, triethyl citrate, N-methyl-2-pyrrolidone, and glycofurol.

22. The composition of claim 1, wherein the hydrophilic porogen comprises one selected from the group consisting of sugars, hydrophilic solid polymers, inorganic salts, cross-linked hydrogels, and combinations thereof.

23. The composition of claim 22, further comprising a mineral.

24. The composition of claim 23, wherein the mineral is incorporated in the viscous gel.

25. The composition of claim 1, which forms a porous scaffold in situ.

26. The composition of claim 25, wherein the porous scaffold has a pore density in a range from 1% to 70% of the total mass of the composition.

27. The composition of claim 25, wherein the porous scaffold has a pore density in a range from 5% to 50% of the total mass of the composition.

28. The composition of claim 25, wherein the porous scaffold has a pore density in a range from 10% to 40% of the total mass of the composition.

29. The composition of claim 25, wherein the porous scaffold has a pore size in a range from 1 to 1,000 microns.

30. The composition of claim 25, wherein the porous scaffold has a pore size in a range from 5 to 500 microns.

31. The composition of claim 25, wherein the porous scaffold has a pore size in a range from 30 to 300 microns.

32. A drug delivery device, comprising:
a composition which forms a porous scaffold in situ, the composition comprising a viscous gel formed from a combination of a biodegradable polymer and a biocompatible solvent and a hydrophilic porogen incorporated in the viscous gel.

33. The drug delivery device of claim 32, wherein the composition further comprises an active agent formulation incorporated in the viscous gel, the active agent formulation comprising at least one active agent.

34. The drug delivery device of claim 32, wherein the composition is contained in a patch.
